SEQUENCE LISTING (7 sequences)

SEQ ID NO: 1 - DNA, 765 bp, Homo sapiens
atgtggcagctgctgctgcctacagctctcctgctgctggtgtccgccggcatgagaaccgaggatctgcctaaggccgtggtgttcctggaaccccagtggtacagagtgctggaaaaggacagcgtgaccctgaagtgccagggcgcctacagccccgaggacaatagcacccagtggttccacaacgagagcctgatcagcagccaggccagcagctacttcatcgacgccgccaccgtggacgacagcggcgagtatagatgccagaccaacctgagcaccctgagcgaccccgtgcagctggaagtgcacatcggatggctgctgctgcaggcccccagatgggtgttcaaagaagaggaccccatccacctgagatgccactcttggaagaacaccgccctgcacaaagtgacctacctgcagaacggcaagggcagaaagtacttccaccacaacagcgacttctacatccccaaggccaccctgaaggactccggctcctacttctgcagaggcctcgtgggcagcaagaacgtgtccagcgagacagtgaacatcaccatcacccagggcctggccgtgtctaccatcagcagctttttcccacccggctaccaggtgtccttctgcctcgtgatggtgctgctgttcgccgtggacaccggcctgtacttcagcgtgaaaacaaacatcagaagcagcacccgggactggaaggaccacaagttcaagtggcggaaggacccccaggacaagtga

SEQ ID NO: 2 - protein, 254 aa, Homo sapiens
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQVSFCLVMVLLFAVDTGLYFSVKTNIRSSTRDWKDHKFKWRKDPQDK

SEQ ID NO: 3 - DNA, 483 bp, Homo sapiens
atgtaccggatgcagctgctgagctgtatcgccctgtctctggccctcgtgaccaacagcgcccctaccagcagcagcaccaagaaaacccagctgcagctggaacatctgctgctggacctgcagatgatcctgaacggcatcaacaactacaagaaccccaagctgacccggatgctgaccttcaagttctacatgcccaagaaggccaccgaactgaaacatctgcagtgcctggaagaggaactgaagcccctggaagaagtgctgaacctggcccagagcaagaacttccacctgaggcccagggacctgatcagcaacatcaacgtgatcgtgctggaactgaaaggcagcgagacaaccttcatgtgcgagtacgccgacgagacagctaccatcgtggaatttctgaaccggtggatcaccttctgccagagcatcatcagcaccctgaccggctccgagaaggacgagctgtga

SEQ ID NO: 4 - protein, 160 aa, Homo sapiens
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGSEKDEL

SEQ ID NO: 5 - protein, 405 aa, Artificial Sequence
  source: /note=“Description of Artificial Sequence Synthetic polypeptide”
MDWIWRILFLVGAATGAHSAQPANIQMTQSPSSVSASVGDRVTITCRASQDISRWLAWYQQKPGKAPKLLIYAASSLQSGVPSRFSGSGSGTDFALTISSLQPEDFATYYCQQADSRFSITFGQGTRLEIKGGGGSGGGGSGGGGSGGGGSEVQLVQSGGGLVQPGGSLRLSCAASGFTFSSYSMNWVRQAPGKGLEWVSYISSSSSTIQYADSVKGRFTISRDNAKNSLYLQMNSLRDEDTAVYYCARGDYYYGMDVWGQGTTVTVSSAAALSNSIMYFSHFVPVFLPAKPTTTPAPRPPTPAPTIASQPLSLRPEACRPAAGGAVHTRGLDFACFWVLVVVGGVLACYSLLVTVAFIIFWVRLKIQVRKAAITSYEKSDGVYTGLSTRNQETYETLKHEKPPQ

SEQ ID NO: 6 - protein, 407 aa, Artificial Sequence
  source: /note=“Description of Artificial Sequence Synthetic polypeptide”
MDWIWRILFLVGAATGAHSAQPADIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLELKRGGGGSGGGGSGGGGSGGGGSEVQLQQSGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTTVTVSSAAALSNSIMYFSHFVPVFLPAKPTTTPAPRPPTPAPTIASQPLSLRPEACRPAAGGAVHTRGLDFACFWVLVVVGGVLACYSLLVTVAFIIFWVRLKIQVRKAAITSYEKSDGVYTGLSTRNQETYETLKHEKPPQ

SEQ ID NO: 7 - protein, 275 aa, Artificial Sequence
  source: /note=“Description of Artificial Sequence Synthetic polypeptide”
MDWIWRILFLVGAATGAHSAQPADIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKSSGGGGSGGGGSGGGGSGGGGSGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSS

(19) United States
(12) Patent Application Publication
(10) Pub. No.: US 2021/0040451 A1
(43) Pub. Date: Feb. 11, 2021

(54) USE OF 5% HUMAN ALBUMIN IN WASH AND HARVEST MEDIA

(71) Applicant: NantKwest, Inc., San Diego, CA (US)

(72) Inventors: Shannyn BESSETTE, San Diego, CA (US); Syed Raza ALI, San Diego, CA (US); Manju SAXENA, San Diego, CA (US)

(21) Appl. No.: 16/964,962

(22) PCT Filed: Jan. 30, 2019
(86) PCT No.: PCT/US2019/015841

§ 371 Date: Jul. 24, 2020

Related U.S. Application Data
(60) Provisional application No. 62/624,624, filed on Jan. 31, 2018.

(51) Int. Cl. C12N 5/0783; A61K 35/17

(52) U.S. Cl. CPC C12N 5/0646; C12N 2500/90; A61K 35/17

(57) ABSTRACT

Provided herein are methods of harvesting NK-92® cells comprising collecting NK-92® cells from a cell culture and washing the collected NK-92® cells in a buffer comprising 1%-5% albumin.

### RELATED APPLICATIONS

This application claims priority to U.S. Provisional Application No. 62/624,624, filed on Jan. 31, 2018. The content of said provisional application is herein incorporated by reference in its entirety for all purposes.

### BACKGROUND

Natural killer (NK) cells are cytotoxic lymphocytes that constitute a major component of the innate immune system. NK cells, generally representing about 10-15% of circulating lymphocytes, bind and kill targeted cells, including virus-infected cells and many malignant cells, non-specifically with regard to antigen and without prior immune sensitization. Herberman et al., Science 214:24 (1981). Killing of targeted cells occurs by inducing cell lysis. NK cells used for this purpose are isolated from the peripheral blood lymphocyte (“PBL”) fraction of blood from the subject, expanded in cell culture in order to obtain sufficient numbers of cells, and then re-infused into the subject. NK cells have been shown to be somewhat effective in both ex vivo therapy and in vivo treatment. However, such therapy is complicated by the fact that not all NK cells are cytolytic and the therapy is specific to the treated patient.

NK-92® cells have previously been evaluated as a therapeutic agent in the treatment of certain cancers. Unlike NK cells, NK-92® is a cytolytic cancer cell line which was discovered in the blood of a subject suffering from a non-Hodgkins lymphoma and then immortalized ex vivo. NK-92® cells lack the major inhibitory receptors that are displayed by normal NK cells, but retain the majority of the activating receptors. NK-92® cells do not, however, attack normal cells nor do they elicit an unacceptable immune rejection response in humans. Characterization of the NK-92® cell line is disclosed, e.g., in WO 1998/49268 and U.S. Pat. No. 8,034,332.

However, the poor efficiency of cell harvesting remains a significant challenge for producing sufficient NK-92® cells for various therapeutic applications. Conventional harvesting procedures typically involve collecting cells from cell culture medium and washing the cells in buffer such as PBS. The multiple washes in PBS cause cell loss and a significant reduction in viability. In some instances, cells are washed in a medium, such as X—VIVO™10; this is also not ideal because the final product formulation requires two additional steps including centrifugation for removal of X-VIVO™10. These steps increase processing time and cause cell stress and cell loss due to the repeated centrifugation steps that are required.

### BRIEF SUMMARY

Provided herein are methods of harvesting NK-92® cells comprising collecting NK-92® cells from a cell culture and washing the collected NK-92® cells by a buffer comprising 1-5% albumin. The NK-92® cells may be those modified to express one or more transgenes, for example, the NK-92® cells can be modified to express a cytokine, a Fc receptor, a chimeric antigen receptor, or a combination thereof.

Optionally, the collecting NK-92® cells comprising centrifuging the NK-92® cells in the cell culture. Optionally, the method further comprise placing the washed NK-92® cells in a infusion bag. Optionally, the wash is performed by centrifuging the cells and then resuspending the cells in the wash buffer. Optionally, the wash is performed at least three times, e.g., four to six times. Optionally, the method recovers at least 80% of the NK-92® cells. Optionally, the viability of the harvested cells is at least 90%.

Optionally, the NK-92® cells that have been harvested have substantially the same cytotoxicity and/or viability as control NK-92® cells that have not been harvested. Optionally, the NK-92® cells that have been harvested have substantially the same cytotoxicity and/or viability as the NK-92® cells before harvesting. Optionally, the NK-92® cells that have been harvested have a cytotoxicity of 80-100% on K562 cells. Optionally, the buffer contains 2-5% albumin, e.g., 3-5% albumin, or 5% albumin. Optionally, the buffer lacks sugar. Optionally, the buffer lacks dextran. Optionally, the centrifugation is by continuous centrifugation. Optionally, the albumin is human plasma albumin or human serum albumin. Optionally, the NK-92® cells express a cytokine, Fc Receptor, a chimeric antigen receptor, or a combination thereof.

The foregoing general description and the following detailed description are exemplary and explanatory and are intended to provide further explanation of the disclosure. Other objects, advantages and novel features will be readily apparent to those skilled in the art.

### BRIEF DESCRIPTION OF THE DRAWINGS

The objects, features and advantages will be more readily appreciated upon reference to the following disclosure when considered in conjunction with the accompanying drawings.

FIG. 1 is a schematic illustration of an exemplary process of harvesting NK-92® cells.

### DETAILED DESCRIPTION

Provided herein are methods of harvesting NK-92® cells using a buffer containing 1-5% albumin, optionally 5% human albumin. After washing, the cells can be directly used for therapeutic applications, such as infusion, without the need for further processing steps or formulation. This advantageously reduces processing times and minimize cell loss and cell stress.

After reading this description, it will become apparent to one skilled in the art how to implement various alternative embodiments and alternative applications. However, not all embodiments are described herein. It will be understood that the embodiments presented here are presented by way of an example only, and not limitation. As such, this detailed description of various alternative embodiments should not be construed to limit the scope or breadth of the disclosure as set forth herein. It is to be understood that the aspects described below are not limited to specific compositions, methods of preparing such compositions, or uses thereof as such may, of course, vary.

### Terminology

Unless defined otherwise, all technical and scientific terms used herein have the same meaning as commonly understood by one of ordinary skill in the art.

In this specification and in the claims that follow, reference will be made to a number of terms that shall be defined to have the following meanings:

The terminology used herein is for the purpose of describing particular embodiments only and is not intended to be limiting. As used herein, the singular forms “a,” “an” and “the” are intended to include the plural forms as well, unless the context clearly indicates otherwise. Thus, for example, reference to “a natural killer cell” includes a plurality of natural killer cells.

All numerical designations, e.g., pH, temperature, time, concentration, amounts, and molecular weight, including ranges, are approximations which are varied (+) or (−) by increments of 0.1 or 1.0, where appropriate. It is to be understood, although not always explicitly stated, that all numerical designations may be preceded by the term “about.”

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

It is also to be understood, although not always explicitly stated, that the reagents described herein are merely exemplary and that equivalents of such are known in the art.

“Optional” or “optionally” means that the subsequently described event or circumstance can or cannot occur, and that the description includes instances where the event or circumstance occurs and instances where it does not.

The term “comprising” is intended to mean that the compositions and methods include the recited elements, but not excluding others. “Consisting essentially of,” when used to define compositions and methods, shall mean excluding other elements of any essential significance to the combination. For example, a composition consisting essentially of the elements as defined herein would not exclude other elements that do not materially affect the basic and novel characteristic(s) of the claims. “Consisting of” shall mean excluding more than trace amount of other ingredients and substantial method steps. Embodiments defined by each of these transition terms are within the scope of the disclosure.

As used herein, “natural killer (NK) cells” are cells of the immune system that kill target cells in the absence of a specific antigenic stimulus, and without restriction according to major histocompatibility complex (MHC) class. Target cells may be cancer or tumor cells. NK cells are characterized by the presence of CD56 and the absence of CD3 surface markers.

For purposes of this invention and unless indicated otherwise, the term NK-92® or “NK-92®” is intended to refer to the original NK-92® cell lines as well as NK-92® cell lines, clones of NK-92® cells, and NK-92® cells that have been modified (e.g., by introduction of exogenous genes). NK-92® cells and exemplary and non-limiting modifications thereof are described in U.S. Pat. Nos. 7,618,817; 8,034,332; 8,313,943; 9,181,322; 9,150,636; and published U.S. application Ser. No. 10/008,955, all of which are incorporated herein by reference in their entireties, and include wild type NK-92®, NK-92®-CD16, NK-92®-CD16-γ, NK-92®-CD16-ζ, NK-92®-CD16(F176V), NK-92® MI, and NK-92® C.I. NK-92® cells are known to persons of ordinary skill in the art, to whom such cells are readily available from NantKwest, Inc.

As used herein, the term “a NK cells” refers to the parental NK-92® cells.

As used herein, the term “ha NK cells” refers to NK-92® cells that have been engineered to express Fc receptor.

As used herein, the term “ta NK cells” refers to NK-92® cells that have been engineered to express a chimeric antigen receptor (CAR) with affinity for a cancer specific antigen, a cancer associated antigen, or a tumor specific antigen. In some embodiments, the tumor specific antigen is HER-2, e.g., human HER-2, and these NK-92® cells are referred to as HER-2 ta NK cells.

As used herein, the term “t-ha NK cells” refers to NK-92® cells that have been engineered to express a chimeric antigen receptor (CAR) with affinity for a cancer specific antigen, a cancer associated antigen, or a tumor specific antigen and to express Fc receptor. In some embodiments, the tumor specific antigen is CD19, e.g., human CD19, and these NK-92® cells are referred to as CD19 t-ha NK cells. In some embodiments, the tumor specific antigen is PD-L1. In some embodiments, the t-ha NK cells express a chimeric antigen receptor PD-L1 CAR that has a sequence of SEQ ID NO: 5. In some embodiments, the t-ha NK cells express a chimeric antigen receptor CD19 CAR that has a sequence of SEQ ID NO: 6. In some embodiments, the t-ha NK cells express a chimeric antigen receptor HER2 CAR that has a sequence of SEQ ID NO: 7.

The term “Fc receptor” refers to a protein found on the surface of certain cells (e.g., natural killer cells) that contribute to the protective functions of the immune cells by binding to part of an antibody known as the Fc region. Binding of the Fc region of an antibody to the Fc receptor (FcR) of a cell stimulates phagocytic or cytotoxic activity of a cell via antibody-mediated phagocytosis or antibody-dependent cell-mediated cytotoxicity (ADCC). FcRs are classified based on the type of antibody they recognize. For example, Fc-gamma receptors (FcγR) bind to the IgG class of antibodies. FcγRIII-A (also called CD16) is a low affinity Fc receptor bind to IgG antibodies and activate ADCC. FcγRIII-A are typically found on NK cells. NK-92® cells do not express FcγRIII-A.

The term “chimeric antigen receptor” (CAR), as used herein, refers to an extracellular antigen-binding domain that is fused to an intracellular signaling domain. CARs can be expressed in T cells or NK cells to increase cytotoxicity. In general, the extracellular antigen-binding domain is a scFv that is specific for an antigen found on a cell of interest. A CAR-expressing NK-92® cell is targeted to cells expressing certain antigens on the cell surface, based on the specificity of the scFv domain. The scFv domain can be engineered to recognize any antigen, including tumor-specific antigens.

The terms “polynucleotide”, “nucleic acid” and “oligonucleotide” are used interchangeably and refer to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, a polynucleotide encompasses both the double-stranded form and each of two complementary single-stranded forms known or predicted to make up the double-stranded form.

The term “expression” refers to the production of a gene product. The term “transient” when referred to expression means a polynucleotide is not incorporated into the genome of the cell.

The term “cytokine” or “cytokines” refers to the general class of biological molecules which effect cells of the immune system. Exemplary cytokines include, but are not limited to, interferons and interleukins (IL), in particular IL-2, IL-12, IL-15, IL-18 and IL-21. In preferred embodiments, the cytokine is IL-2.

As used herein, the term “vector” refers to a non-chromosomal nucleic acid comprising an intact replicon such that the vector may be replicated when placed within a permissive cell, for example by a process of transformation. A vector may replicate in one cell type, such as bacteria, but have limited ability to replicate in another cell, such as mammalian cells. Vectors may be viral or non-viral. Exemplary non-viral vectors for delivering nucleic acid include naked DNA; DNA complexed with cationic lipids, alone or in combination with cationic polymers; anionic and cationic liposomes; DNA-protein complexes and particles comprising DNA condensed with cationic polymers such as heterogeneous polylysine, defined-length oligopeptides, and polyethylene imine, in some cases contained in liposomes; and the use of ternary complexes comprising a virus and polylysine-DNA.

As used herein, the term “substantially the same”, used interchangeably with the term “comparable”, or “similar”, when referring to cytotoxicity, viability or cell recovery, refers to the that the two measurements of cytotoxicity, viability or cell recovery are no more than 15% different, no more than 10%, no more than 8%, or no more than 5% different from each other.

As used herein, the terms “cytotoxic” when used to describe the activity of effector cells such as NK cells, relates to killing of target cells by any of a variety of biological, biochemical, or biophysical mechanisms.

As used herein, the term “harvesting” refers to separating and collecting cells from their culture medium and preparing the cells for therapeutic applications. Harvesting comprises washing cells with a suitable buffer, e.g., 5% albumin, and optionally resuspending cells in a buffer that is suitable for intended applications, e.g., for infusion.

As used herein, the term “recovery” refers to relative amount of the cells obtained after the harvesting process is completed as compared to the number of cells entering the harvesting process. In some cases, recovery is expressed as a percentage, for example, when continuous centrifugation is used as a means for harvesting cells, recovery can be expressed as the following equation:

Recovery=the amount of cells recovered from continuous centrifugation/amount of cells entered into continuous centrifugation.

Titles or subtitles may be used in the specification for the convenience of a reader, which are not intended to influence the scope of the present disclosure. Additionally, some terms used in this specification are more specifically defined below.

### Albumin

Albumin is a protein supplement in cell culture used to deliver unesterified fatty acids into and from cells; Albumin can be derived from human or non-human sources, for example, human or bovine. Human albumin can be derived from human serum (“human serum albumin”) or human plasma (“human plasma albumin”), or can be synthesized in vitro, e.g., by expressing a gene (e.g., sequence of NM_000477) encoding the human albumin. To date, albumin, especially albumin derived from human has not been used for washing cells during harvesting because it is relatively costly as compared to other wash buffers, such as PBS or growth media, such as X VIVO™ 10. Human Albumin is commercially available, for example, from CSL Behring.

### Methods of Harvesting NK-92® Cells

Growing NK-92® cells typically starts from thawing frozen NK-92® cells and seeding them in a container with a suitable medium. Cells are allowed to recover until the cell viability reaches a certain value, for example, greater than 85%. Cells are then expanded in a vessel, e.g., a G-Rex flask, to a desirabed cell density, for example, a density that is equal to or less than 1.2×10⁶ cells/mL. The cell culture from the vessel is then collected and used to inoculate one or more larger culture vessels. Commonly used such larger culture vessels include Xuri bags, which can have a volume of at least 2 liters, at least 10 liters, or at least 50 liters. Transfer of cells between the different vessels can be performed using means well known in the art, e.g., using a pump or a gravity feed, performed under sterile conditions.

NK-92® cells so produced can be harvested by centrifugation. Optionally the centrifugation is performed in a continuous centrifuge that are aseptically attached to the culture vessel, e.g., the Xuri bags, that is at the end of the expansion process. Continuous centrifugation refers to a centrifugation of a duration of 45-60 min, depending on the cell culture volume, to concentrate the cells, followed by a cell wash of at least 1 min, at least 3 min, or at least 5 min. The culture supernatant is then removed and the cells are resuspended in a wash buffer comprising 1-5% albumin, e.g., 2-5%, 3-5%, or 4-5%, preferably 5% albumin. Optionally, the wash can be repeated for at least two times, at least three times, e.g., 4-6 times. After the final wash, the mixture containing the cells and wash buffer can be centrifuged again and the cells are collected and processed for therapeutic applications.

In addition to albumin, the wash buffer may also comprise 1-10 mg/mL sodium, e.g., 3-5 mg/mL sodium, or 3.2 mg/mL sodium. Optionally, the wash buffer lacks sugar, e.g., dextran. Optionally, the wash buffer lacks dextran-40.

The method of harvesting can recover 80 to 100% of NK-92® cells, e.g., 85-99%, or 89-99% of NK-92® cells. The yield of havesting can be assessed using standard cell counting procedure, e.g., a trypan blue dye-exclusion method or a Nucleocounter NC-200 method. Optionally, the method of harvesting using the methods disclosed herein can recover substantially the same amount of NK-92® cells as the method of harvesting using X-VIVO10 medium.

NK-92® cells harvested using 1-5% albumin as disclosed herein may have substantially the same cytotoxicity as control NK-92® cells that have been grown in the same condition but have not been harvested. The control cells can be, for example, the NK92® cells from the G-Rex flask. The NK-92® cells harvested using the method disclosed herein can also have substantially the same cytotoxicity as NK-92® cells that have been harvested in X-VIVO10 medium. The NK-92® cells that have been harvested using the methods disclosed herein may have substantially the same cytotoxicity as the NK-92® cells before harvesting. See Table 2.

Cytotoxicity of NK-92® cells can be reflected by their direct cytotoxicity or ADCC. Direct cytotoxicity of the produced NK-92® cells, the ability to target and kill aberrant cells, such as virally infected and tumorigenic cells, can be assessed by methods well known in the art, for example, a ⁵¹Cr release assay, (Gong et al. (1994)) using the procedure described by Klingemann et al. (Cancer Immunol. Immunother. 33:395-397 (1991)). Briefly, ⁵¹Cr-labeled target cells are mixed with NK-92® cells and are lysed. The percentage of specific cytotoxicity can be calculated based on the amount of released ⁵¹Cr. See Patent Pub. No. US20020068044.

Alternatively, direct cytotoxicity of the produced NK-92® cells can also be assessed using a calcein release assay. For example, the NK-92® cells (referred to as the effector in the assay) can be mixed with the calcein loaded target cells (referred to as target in the assay) at certain ratios. After incubation for a period of time, the calcein released from the target cells can be assessed, e.g., by a fluorescence plate reader. The ratio of the effector and target used in the assay may vary, optionally the effector:target ratio may be 20:1, 15:1, 10:1, 8:1, or 5:1; preferably the effector: target ratio is 10:1. The target cells can be any cells that express MEW molecules that can be recognized by the NK-92® cells, for example, K562 cells, or BT-474 cells. The values of cytotoxicity of NK-92® cells may vary depending on the type of target cells used as well as the effector:target ratio. In general, the NK-92® cells produced using the methods described herein can have a cytotoxicity of 60-100%, e.g., 70-100% or 80-100%. In some cases, a NK cells may have a cytotoxicity of 80-100% when using K562 cells as the target cells, e.g., 82-100%, 85-100%, 87-100%, 88-100%, or 89-100%, by a calcein release assay.

Optionally, the cytotoxicity of NK-92® cells, e.g., ha NK cells, that is assessed is the antibody dependent cytotoxicity (ADCC). Methods for measuring the ADCC of NK-92® cells are similar to the methods of measuring direct cytotoxicity as described above except that an antibody that can recognize the target cell is added. The Fc receptor of the NK cells recognizes the cell-bound antibodies and triggers cytolytic reaction and killing the target cells. In one illustrative example, the ha NK cells can be incubated with Rituxan (an antibody) and Ramos (target cells) and killing of the Ramos cells can be measured by the release of internal components of the target cells, e.g., ⁵¹Cr or calcein, as described above.

### NK-92® Cells

The NK-92® cells that can be cultured using the methods disclosed herein include a NK cells, ha NK cells, ta NK and t-ha NK cells, which are further described below.

The NK-92® cell line is a unique cell line that was discovered to proliferate in the presence of interleukin 2 (IL-2). Gong et al., Leukemia 8:652-658 (1994). These cells have high cytolytic activity against a variety of cancers. The NK-92® cell line is a homogeneous cancerous NK cell population having broad anti-tumor cytotoxicity with predictable yield after expansion. Phase I clinical trials have confirmed its safety profile. NK-92® was discovered in the blood of a subject suffering from a non-Hodgkins lymphoma and then immortalized ex vivo. NK-92® cells are derived from NK cells, but lack the major inhibitory receptors that are displayed by normal NK cells, while retaining the majority of the activating receptors. NK-92® cells do not, however, attack normal cells nor do they elicit an unacceptable immune rejection response in humans. Characterization of the NK-92® cell line is disclosed in WO 1998/49268 and U.S. Patent Application Publication No. 2002-0068044.

The NK-92® cell line is found to exhibit the CD56^{bright}, CD2, CD7, CD11a, CD28, CD45, and CD54 surface markers. It furthermore does not display the CD1, CD3, CD4, CD5, CD8, CD10, CD14, CD16, CD19, CD20, CD23, and CD34 markers. Growth of NK-92® cells in culture is dependent upon the presence of recombinant interleukin 2 (rIL-2), with a dose as low as 1 IU/mL being sufficient to maintain proliferation. IL-7 and IL-12 do not support long-term growth, nor do other cytokines tested, including IL-1α, IL-6, tumor necrosis factor α, interferon α, and interferon γ. NK-92® has high cytotoxicity even at a low effector:target (E:T) ratio of 1:1. Gong, et al., supra. NK-92® cells are deposited with the American Type Culture Collection (ATCC), designation CRL-2407.

Heretofore, studies on endogenous NK cells have indicated that IL-2 (1000 IU/mL) is critical for NK cell activation during shipment, but that the cells need not be maintained at 37° C. and 5% carbon dioxide. Koepsell, et al., Transfusion 53:398-403 (2013).

Modified NK-92® cells are known and include, but are not limited to, those described in, e.g., U.S. Pat. Nos. 7,618,817, 8,034,332, and 8,313,943, US Patent Application Publication No. 2013/0040386, all of which are incorporated herein by reference in their entireties, such as wild type NK-92®, NK-92®-CD16, NK-92®-CD16-γ, NK-92®-CD16-ζ, NK-92®-CD16(F157V), NK-92® mi and NK-92® ci.

Although NK-92® cells retain almost all of the activating receptors and cytolytic pathways associated with NK cells, they do not express CD16 on their cell surfaces. CD16 is an Fc receptor which recognizes and binds to the Fc portion of an antibody to activate NK cells for antibody-dependent cellular cytotoxicity (ADCC). Due to the absence of CD16 receptors, NK-92® cells are unable to lyse target cells via the ADCC mechanism and, as such, cannot potentiate the anti-tumor effects of endogenous or exogenous antibodies (i.e., Rituximab and Herceptin).

Studies on endogenous NK cells have indicated that IL-2 (1000 IU/mL) is critical for NK cell activation during shipment, but that the cells need not be maintained at 37° C. and 5% carbon dioxide. Koepsell, et al., Transfusion 53:398-403 (2013). However, endogenous NK cells are significantly different from NK-92® cells, in large part because of their distinct origins: NK-92® is a cancer-derived cell line, whereas endogenous NK cells are harvested from a donor (or the patient) and processed for infusion into a patient. Endogenous NK cell preparations are heterogeneous cell populations, whereas NK-92® cells are a homogeneous, clonal cell line. NK-92® cells readily proliferate in culture while maintaining cytotoxicity, whereas endogenous NK cells do not. In addition, an endogenous heterogeneous population of NK cells does not aggregate at high density. Furthermore, endogenous NK cells express Fc receptors, including CD-16 receptors that are not expressed by NK-92® cells.

### Fc Receptors

Fc receptors bind to the Fc portion of antibodies. Several Fc receptors are known, and differ according to their preferred ligand, affinity, expression, and effect following binding to the antibody.

TABLE 1
Illustrative Fc receptors

| | Principal | Affinity | | |
|---|---|---|---|---|
| Receptor | antibody | for | | Effect following binding |
| name | ligand | ligand | Cell distribution | to antibody |
| FcγRI (CD64) | IgG1 and | High | Macrophages | Phagocytosis |
| | IgG3 | (Kd ~10⁻⁹ M) | Neutrophils | Cell activation |
| | | | Eosinophils | Activation of respiratory |
| | | | Dendritic cells | burst |
| | | | | Induction of microbe |
| | | | | killing |
| FcγRIIA (CD32) | IgG | Low | Macrophages | Phagocytosis |
| | | (Kd > | Neutrophils | Degranulation (eosinophils) |
| | | 10⁻⁷ M) | Eosinophils | |
| | | | Platelets | |
| | | | Langerhans cells | |
| FcγRIIB1 (CD32) | IgG | Low | B Cells | No phagocytosis |
| | | (Kd > | Mast cells | Inhibition of cell activity |
| | | 10⁻⁷ M) | | |
| FcγRIIB2 (CD32) | IgG | Low | Macrophages | Phagocytosis |
| | | (Kd > | Neutrophils | Inhibition of cell activity |
| | | 10⁻⁷ M) | Eosinophils | |
| FcγRIIIA (CD16a) | IgG | Low | NK cells | Induction of antibody- |
| | | (Kd > | Macrophages (certain | dependent cell-mediated |
| | | 10⁻⁶ M) | tissues) | cytotoxicity (ADCC) |
| | | | | Induction of cytokine |
| | | | | release by macrophages |
| FcγRIIIB (CD16b) | IgG | Low | Eosinophils | Induction of microbe |
| | | (Kd > | Macrophages | killing |
| | | 10⁻⁶ M) | Neutrophils | |
| | | | Mast cells | |
| | | | Follicular dendritic | |
| | | | cells | |
| FcεRI | IgE | High | Mast cells | Degranulation |
| | | (Kd ~10⁻¹⁰ M) | Eosinophils | Phagocytosis |
| | | | Basophils | |
| | | | Langerhans cells | |
| | | | Monocytes | |
| FcεRII (CD23) | IgE | Low | B cells | Possible adhesion molecule |
| | | (Kd > | Eosinophils | IgE transport across human |
| | | 10⁻⁷ M) | Langerhans cells | intestinal epithelium |
| | | | | Positive-feedback |
| | | | | mechanism to enhance |
| | | | | allergic sensitization (B |
| | | | | cells) |
| FcαRI (CD89) | IgA | Low | Monocytes | Phagocytosis |
| | | (Kd > | Macrophages | Induction of microbe |
| | | 10⁻⁶ M) | Neutrophils | killing |
| | | | Eosinophils | |
| Fcα/μR | IgA and IgM | High for | B cells | Endocytosis |
| | | IgM, | Mesangial cells | Induction of microbe |
| | | Mid for | Macrophages | killing |
| | | IgA | | |
| FcRn | IgG | | Monocytes | Transfers IgG from a |
| | | | Macrophages | mother to fetus through the |
| | | | Dendritic cells | placenta |
| | | | Epithelial cells | Transfers IgG from a |
| | | | Endothelial cells | mother to infant in milk |
| | | | Hepatocytes | Protects IgG from |
| | | | | degradation |

In some embodiments NK-92® cells are modified to express an Fc receptor protein on the cell surface.

In some embodiments, the Fc receptor is CD16. A representative amino acid sequence encoding CD16 is shown in SEQ ID NO:2. A representative polynucleotide sequence encoding CD16 is shown in SEQ ID NO:1. In some embodiments, NK-92® cells are modified by introducing a polynucleotide encoding a CD16 polypeptide has at least about 70% polynucleotide sequence identity with a polynucleotide sequence encoding a full-length, including signal peptide, naturally occurring CD16 that has a phenylalanine at position 176 of the full-length CD16. In some embodiments, a polynucleotide encoding a CD16 polypeptide has at least about 70% polynucleotide sequence identity with a polynucleotide sequence encoding a full-length, including the signal peptide, naturally occurring CD16 that has a valine at position 176.

Homologous polynucleotide sequences include those that encode polypeptide sequences coding for variants of CD16. In some embodiments, homologous CD16 polynucleotides may be about 150 to about 700, about 750, or about 800 polynucleotides in length, although CD16 variants having more than 700 to 800 polynucleotides are within the scope of the disclosure.

In other examples, cDNA sequences having polymorphisms that change the CD16 amino acid sequences are used to modify the NK-92® cells, such as, for example, the allelic variations among individuals that exhibit genetic polymorphisms in CD16 genes. In other examples, CD16 genes from other species that have a polynucleotide sequence that differs from the sequence of human CD16 are used to modify NK-92® cells.

In examples, variant polypeptides are made using methods known in the art such as oligonucleotide-mediated (site-directed) mutagenesis, alanine scanning, and PCR mutagenesis. Site direct mutagenesis (Carter, 1986; Zoller and Smith, 1987), cassette mutagenesis, restriction selection mutagenesis (Wells et al., 1985) or other known techniques can be performed on the cloned DNA to produce CD16 variants (Ausubel, 2002; Sambrook and Russell, 2001).

Conservative substitutions in the amino acid sequence of human CD16 polypeptide, whereby an amino acid of one class is replaced with another amino acid of the same class, fall within the scope of the disclosed CD16 variants as long as the substitution does not materially alter the activity of the polypeptide. Conservative substitutions are well known to one of skill in the art. Non-conservative substitutions that affect (1) the structure of the polypeptide backbone, such as a β-sheet or α-helical conformation, (2) the charge, (3) the hydrophobicity, or (4) the bulk of the side chain of the target site can modify CD16 polypeptide function or immunological identity. Non-conservative substitutions entail exchanging a member of one of these classes for another class. Substitutions may be introduced into conservative substitution sites or more preferably into non-conserved sites.

In some embodiments, CD16 polypeptide variants are at least 200 amino acids in length and have at least 70% amino acid sequence identity, or at least 80%, or at least 90% identity to SEQ ID NO:1 or SEQ ID NO:2. In some embodiments, CD16 polypeptide variants are at least 225 amino acid in length and have at least 70% amino acid sequence identity, or at least 80%, or at least 90% identity to SEQ ID NO:1 or SEQ ID NO:2.

In some embodiments a nucleic acid encoding a CD16 polypeptide may encode a CD16 fusion protein. A CD16 fusion polypeptide includes any portion of CD16 or an entire CD16 fused with a non-CD16 polypeptide. In some embodiment, a fusion polypeptide may be created in which a heterologous polypeptide sequence is fused to the C-terminus of CD16 or is positioned internally in the CD16. Typically, up to about 30% of the CD16 cytoplasmic domain may be replaced. Such modification can enhance expression or enhance cytotoxicity (e.g., ADCC responsiveness). In other examples, chimeric proteins, such as domains from other lymphocyte activating receptors, including but not limited to Ig-a, Ig-B, CD3-e, CD3-d, DAP-12 and DAP-10, replace a portion of the CD16 cytoplasmic domain.

Fusion genes can be synthesized by conventional techniques, including automated DNA synthesizers and PCR amplification using anchor primers that give rise to complementary overhangs between two consecutive gene fragments that can subsequently be annealed and re-amplified to generate a chimeric gene sequence (Ausubel, 2002). Many vectors are commercially available that facilitate sub-cloning CD16 in-frame to a fusion moiety.

### Chimeric Antigen Receptor

As described herein, NK-92® cells are further engineered to express a chimeric antigen receptor (CAR) on the cell surface. Optionally, the CAR is specific for a tumor-specific antigen. Tumor-specific antigens are described, by way of non-limiting example, in US 2013/0189268; WO 1999024566 A1; U.S. Pat. No. 7,098,008; and WO 2000020460 A1, each of which is incorporated herein by reference in its entirety. Tumor-specific antigens include, without limitation, NKG2D, CS1, GD2, CD138, EpCAM, EBNA3C, GPA7, CD244, CA-125, ETA, MAGE, CAGE, BAGE, HAGE, LAGE, PAGE, NY-SEO-1, GAGE, CEA, CD52, CD30, MUC5AC, c-Met, EGFR, FAB, WT-1, PSMA, NY-ESO1, AFP, CEA, CTAG1B, CD19 and CD33. Additional non-limiting tumor-associated antigens, and the malignancies associated therewith, can be found in Table 2.

TABLE 2
Tumor-Specific Antigens and Associated Malignancies

| Target Antigen | Associated Malignancy |
|---|---|
| α-Folate Receptor | Ovarian Cancer |
| CAIX | Renal Cell Carcinoma |
| CD19 | B-cell Malignancies |
| | Chronic lymphocytic leukemia (CLL) |
| | B-cell CLL (B-CLL) |
| | Acute lymphoblastic leukemia (ALL); ALL |
| | post Hematopoietic stem cell transplantation |
| | (HSCT) |
| | Lymphoma; Refractory Follicular |
| | Lymphoma; B-cell non-Hodgkin lymphoma |
| | (B-NHL) |
| | Leukemia |
| | B-cell Malignancies post-HSCT |
| | B-lineage Lymphoid Malignancies post |
| | umbilical cord blood transplantation |
| | (UCBT) |
| CD19/CD20 | Lymphoblastic Leukemia |
| CD20 | Lymphomas |
| | B-Cell Malignancies |
| | B-cell Lymphomas |
| | Mantle Cell Lymphoma |
| | Indolent B-NHL |
| | Leukemia |
| CD22 | B-cell Malignancies |
| CD30 | Lymphomas; Hodgkin Lymphoma |
| CD33 | AML |
| CD44v7/8 | Cervical Carcinoma |
| CD138 | Multiple Myeloma |
| CD244 | Neuroblastoma |
| CEA | Breast Cancer |
| | Colorectal Cancer |
| CS1 | Multiple Myeloma |
| EBNA3C | EBV Positive T-cells |
| EGP-2 | Multiple Malignancies |
| EGP-40 | Colorectal Cancer |
| EpCAM | Breast Carcinoma |
| Erb-B2 | Colorectal Cancer |
| | Breast Cancer and Others |
| | Prostate Cancer |
| Erb-B 2,3,4 | Breast Cancer and Others |
| FBP | Ovarian Cancer |
| Fetal Acetylcholine | Rhabdomyosarcoma |
| Receptor | |
| GD2 | Neuroblastoma |
| GD3 | Melanoma |
| GPA7 | Melanoma |
| Her2 | Breast Carcinoma |
| | Ovarian Cancer |
| | Tumors of Epithelial Origin |
| Her2/new | Medulloblastoma |
| | Lung Malignancy |
| | Advanced Osteosarcoma |
| | Glioblastoma |
| IL-13R-a2 | Glioma |
| | Glioblastoma |
| | Medulloblastoma |
| KDR | Tumor Neovasculature |
| k-light chain | B-cell Malignancies |
| | B-NHL, CLL |
| LeY | Carcinomas |
| | Epithelial Derived Tumors |
| L1 Cell Adhesion Molecule | Neuroblastoma |
| MAGE-A1 | Melanoma |
| Mesothelin | Various Tumors |
| MUC1 | Breast Cancer; Ovarian Cancer |
| NKG2D Ligands | Various Tumors |
| Oncofetal Antigen (h5T4) | Various Tumors |
| PSCA | Prostate Carcinoma |
| PSMA | Prostate/Tumor Vasculature |
| TAA Targeted by mAb IgE | Various Tumors |
| TAG-72 | Adenocarcinomas |
| VEGF-R2 | Tumor Neovasculature |

In some embodiments, the CAR targets CD19, CD33 or CSPG-4.

In examples, variant polypeptides are made using methods known in the art such as oligonucleotide-mediated (site-directed) mutagenesis, alanine scanning, and PCR mutagenesis. Site direct mutagenesis (Carter, 1986; Zoller and Smith, 1987), cassette mutagenesis, restriction selection mutagenesis (Wells et al., 1985) or other known techniques can be performed on the cloned DNA to produce CD16 variants (Ausubel, 2002; Sambrook and Russell, 2001).

Optionally, the CAR targets an antigen associated with a specific cancer type. Optionally, the cancer is selected from the group consisting of leukemia (including acute leukemias (e.g., acute lymphocytic leukemia, acute myelocytic leukemia (including myeloblastic, promyelocytic, myelomonocytic, monocytic, and erythroleukemia)) and chronic leukemias (e.g., chronic myelocytic (granulocytic) leukemia and chronic lymphocytic leukemia), polycythemia vera, lymphomas (e.g., Hodgkin's disease and non-Hodgkin's disease), multiple myeloma, Waldenstrom's macroglobulinemia, heavy chain disease, solid tumors including, but not limited to, sarcomas and carcinomas such as fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, menangioma, melanoma, neuroblastoma and retinoblastoma.

In some embodiments, a polynucleotide encoding a CAR is mutated to alter the amino acid sequence encoding for CAR without altering the function of the CAR. For example, polynucleotide substitutions leading to amino acid substitutions at “non-essential” amino acid residues can be made in the CARs disclosed above. CARs can be engineered as described, for example, in Patent Publication Nos. WO 2014039523; US 20140242701; US 20140274909; US 20130280285; and WO 2014099671, each of which is incorporated herein by reference in its entirety. Optionally, the CAR is a CD19 CAR, a CD33 CAR or CSPG-4 CAR.

### Additional Modifications—Cytokines

The cytotoxicity of NK-92® cells is dependent on the presence of cytokines (e.g., interleukin-2 (IL-2). The cost of using exogenously added IL-2 needed to maintain and expand NK-92® cells in commercial scale culture is significant. The administration of IL-2 to human subjects in sufficient quantity to continue activation of NK-92® cells would cause adverse side effects.

In some embodiments, FcR-expressing NK-92® cells are further modified to express at least one cytokine and a suicide gene. In specific embodiments, the at least one cytokine is IL-2, IL-12, IL-15, IL-18, IL-21 or a variant thereof. In preferred embodiments, the cytokine is IL-2. A representative nucleic acid encoding IL-2 is shown in SEQ ID NO:3 and a representative polypeptide of IL-2 is shown in SEQ ID NO:4. In certain embodiments the IL-2 is a variant that is targeted to the endoplasmic reticulum.

In one embodiment, the IL-2 is expressed with a signal sequence that directs the IL-2 to the endoplasmic reticulum. Not to be bound by theory, but directing the IL-2 to the endoplasmic reticulum permits expression of IL-2 at levels sufficient for autocrine activation, but without releasing IL-2 extracellularly. See Konstantinidis et al “Targeting IL-2 to the endoplasmic reticulum confines autocrine growth stimulation to NK-92® cells” Exp Hematol. 2005 February; 33(2):159-64. Continuous activation of the FcR-expressing NK-92® cells can be prevented, e.g., by the presence of the suicide gene.

### Additional Modifications—Suicide Gene

The term “suicide gene” is one that allows for the negative selection of the cells. A suicide gene is used as a safety system, allowing the cells expressing the gene to be killed by introduction of a selective agent. This is desirable in case the recombinant gene causes a mutation leading to uncontrolled cell growth. A number of suicide gene systems have been identified, including the herpes simplex virus thymidine kinase (TK) gene, the cytosine deaminase gene, the varicella-zoster virus thymidine kinase gene, the nitroreductase gene, the Escherichia coli gpt gene, and the E. coli Deo gene (also see, for example, Yazawa K, Fisher W E, Brunicardi F C: Current progress in suicide gene therapy for cancer. World J. Surg. 2002 July; 26(7):783-9). As used herein, the suicide gene is active in NK-92® cells. Typically, the suicide gene encodes for a protein that has no ill-effect on the cell but, in the presence of a specific compound, will kill the cell. Thus, the suicide gene is typically part of a system.

In one embodiment, the suicide gene is the thymidine kinase (TK) gene. The TK gene may be a wild-type or mutant TK gene (e.g., tk30, tk75, sr39tk). Cells expressing the TK protein can be killed using ganciclovir.

In another embodiment, the suicide gene is Cytosine deaminase which is toxic to cells in the presence of 5-fluorocytosine. Garcia-Sanchez et al. “Cytosine deaminase adenoviral vector and 5-fluorocytosine selectively reduce breast cancer cells 1 million-fold when they contaminate hematopoietic cells: a potential purging method for autologous transplantation.” Blood 1998 July 15; 92(2):672-82.

In another embodiment, the suicide gene is cytochrome P450 which is toxic in the presence of ifosfamide, or cyclophosphamide. See e.g., Touati et al. “A suicide gene therapy combining the improvement of cyclophosphamide tumor cytotoxicity and the development of an anti-tumor immune response.” Curr Gene Ther. 2014; 14(3):236-46.

In another embodiment, the suicide gene is iCas9. Di Stasi, (2011) “Inducible apoptosis as a safety switch for adoptive cell therapy.” N Engl J Med 365: 1673-1683. See also Morgan, “Live and Let Die: A New Suicide Gene Therapy Moves to the Clinic” Molecular Therapy (2012); 20: 11-13. The iCas9 protein induces apoptosis in the presence of a small molecule AP1903. AP1903 is biologically inert small molecule, that has been shown in clinical studies to be well tolerated, and has been used in the context of adoptive cell therapy.

In one embodiment, the modified NK-92® cells are irradiated prior to administration to the patient. Irradiation of NK-92® cells is described, for example, in U.S. Pat. No. 8,034,332, which is incorporated herein by reference in its entirety. In one embodiment, modified NK-92® cells that have not been engineered to express a suicide gene are irradiated.

### Transgene Expression

Transgenes (e.g., CD19 CAR and CD16) can be engineered into an expression vector by any mechanism known to those of skill in the art. Transgenes may be engineered into the same expression vector or a different expression vector. In preferred embodiments, the transgenes are engineered into the same vector.

In some embodiments, the vector allows incorporation of the transgene(s) into the genome of the cell. In some embodiments, the vectors have a positive selection marker. Positive selection markers include any genes that allow the cell to grow under conditions that would kill a cell not expressing the gene. Non-limiting examples include antibiotic resistance, e.g., geneticin (Neo gene from Tn5).

Any number of vectors can be used to express the Fc receptor and/or the CAR. In some embodiments, the vector is a plasmid. In one embodiment, the vector is a viral vector. Viral vectors include, but are not limited to, retroviral vectors, adenoviral vectors, adeno-associated viral vectors, herpes simplex viral vectors, pox viral vectors, and others.

Transgenes can be introduced into the NK-92® cells using any transfection method known in the art, including, by way of non-limiting example, infection, electroporation, lipofection, nucleofection, or “gene-gun.”

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutations of these compounds may not be explicitly disclosed, each is specifically contemplated and described herein. For example, if a method is disclosed and discussed and a number of modifications that can be made to a number of molecules including the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

### EXAMPLES

The following examples are for illustrative purposes only and should not be interpreted as limitations. There are a variety of alternative techniques and procedures available to those of skill in the art which would similarly permit one to successfully perform the examples below.

### Example 1: Washing Cells Using 5% of Human Albumin Increases Efficiency of Cell Havesting

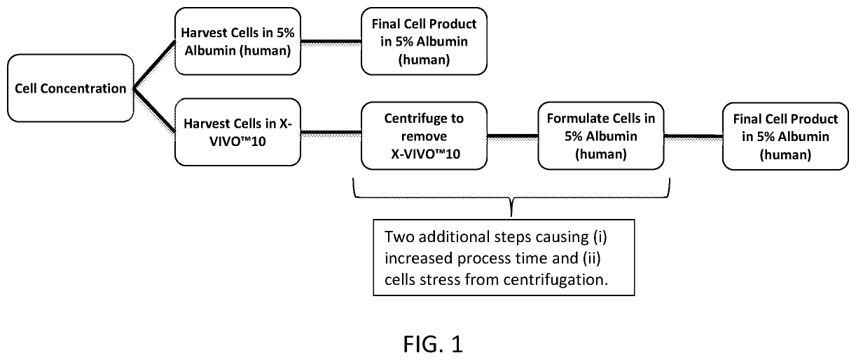

FIG. 1 shows the processes of harvesting modified NK-92® (HER2.ta NK) cells from large bioreactors using either X-VIVO™10 or 5% HA (human albumin). Although cells can be concentrated using both methods, harvesting in X-VIVO™10 requires two additional steps that result in increased process time and cell stress and loss due to centrifugation. Harvesting cells in 5% HUMAN ALBUMIN simplifies the process and improves harvesting efficiency.

### Example 2: NK-92® Cell Viability after being Thawed in 5% Human Albumin

Frozen modified NK-92® (HER2.ta NK) cells were thawed in a 37° C. water bath. 100 μL of the thawed cells were added to 900 μL of the X-VIVO™10 medium, 5% Human Albumin, and PBS, respectively. Cell viability was measured using a Nucleocounter NC-200™ and shown in Table 3.

TABLE 3
Cell viability after being thawed in 5% HUMAN ALBUMIN

| | | | % |
|---|---|---|---|
| Experiment | Study Number | Thaw Media | Viability |
| Thaw of | NKSTUDYTP_028 | X-VIVO ™10 | 94.4 |
| Frozen | | 5% Human Albumin | 86.5 |
| HER2.taNK cells | | PBS | 74.6 |

The results show that cells thawed in 5% Human Albumin had 86.5% viability, which although not as high as X-VIVO™10 (94.4%), was significantly higher than the viability of cells thawed in PBS (74.6%). Cells thawed in 5% Human Albumin and cells thawed in X-VIVO™10 were transferred to respective G-Rex flasks in growth media for expansion, andfurther expanded in 25 L growth media and 10 L growth media in Xuri bags, respectively.

### Example 3: Cytotoxicity of Modified NK-92® (HER2.Ta NK) Cells on BT-474 Target Cells

Modified NK-92® (HER2.ta NK) cells grown in Xuri bags were collected and washed with either X-VIVO™10 medium or 5% Human Albumin using continuous centrifugation. For each group, cytotoxicity assays were performed on cells that were used to feed the continuous centrifuge (i.e., cells that had not undergone harvesting process, referred to as the pre-harvest sample); cells that exit the continuous centrifuge (i.e., cells that had completed the harvesting process, referred to as the post-harvest sample); and cells from the G-rex flask, which had not undergone the harvesting process and were served as control cells. To assess cytotoxicity, cells were mixed with the calcein-loaded target cells at the Effector: Target ratio of 10:1. Calcein release was assessed by fluorescence plate reader post 3 hours of co-incubation. Cytotoxicity was determined using a calcein-release assay and was expressed as the mean±standard deviation percentage (%) of calcein release. The Effector: Target ratio was 10:1 and samples were assayed in triplicates.

The results show that the cytotoxicity of the modified NK-92® (HER2.ta NK) cells harvested using 5% Human Albumin as wash buffer, which was 88±10%, was substantially the same as that of HER2.ta NK cells that were harvested using X-VIVO™ 10 (90±4%). The cytotoxicity of the HER2.ta NK cells harvested using 5% Human Albumin was also substantially the same as the cytotoxicity of the control HER2.ta NK cells, i.e., the cells in the G-Rex flask, which was 90±5%. Further, using 5% Human Albumin as cell wash medium did not impair cytotoxicity of the HER2.ta NK cells, as reflected in that the cytotoxicity of the pre-harvest sample and the cytotoxicity of the post-harvest sample were also substantially similar. See Table 4

TABLE 4
Cytotoxicity of HER2.taNK cells on BT-474 target cells

| | Wash and | | % |
|---|---|---|---|
| Experiment | Harvest Media | Test Sample | Cytotoxicity |
| HER2.taNK | X-VIVO ™ 10 | Pre-harvest | 89 ± 2 |
| cells vs | | Post-harvest | 90 ± 4 |
| BT-474 cells | | G-Rex (reference | 89 ± 1 |
| | | control) | |
| HER2.taNK | 5% Albumin | Pre-harvest | 86 ± 4 |
| cells vs | (Human) | Post-harvest | 88 ± 10 |
| BT-474 cells | | G-Rex (reference | 90 ± 5 |
| | | control) | |

The results show that the cytotoxicity of the modified NK-92® (CD19 t-ha NK and PD-L1 t-ha NK) cells harvested using 5% Albumin (Human) as wash buffer (post-harvest), against tumor cells of different origin were comparable to the cytotoxicity of the cells taken directly from bioreactor (pre-harvest) as well as the reference control cells i.e., the cells in the G-Rex flask. See Table 5.

TABLE 5
Cytotoxicity of modified NK-92 ® cells against tumor cells of different origin

| Modified NK-92 ® | | | | % |
|---|---|---|---|---|
| Effector Cells | Tumor Type | Study Date | Test Sample | Cytotoxicity¹ |
| CD19 t-haNK | B Cell Lymphoma | NKSTUDYTP_100 | Reference | 92 ± 7 |
| Cells | SUP-B15 cells | 13 Jul. 2018 | Pre-harvest | 102 ± 7 |
| | | | Post-harvest | 99 ± 3 |
| | Leukemia Cells | NKSTUDYTP_100 | Reference | 86 ± 7 |
| | K562 cells | 13 Jul. 2018 | Pre-harvest | 72 ± 8 |
| | | | Post-harvest | 81 ± 5 |
| PD-L1 t-haNK | Breast Tumor Cells | NKSTUDYTP_095 | Reference | 87 ± 3 |
| Cells | MDA-MB-231 cells | 11 Jul. 2018 | Pre-harvest | 87 ± 1 |
| | (Triple Negative) | | Post-harvest | 86 ± 4 |

¹ModifiedNK-92 ® cells were centrifuged and harvested in 5% HA. Cells were mixed with the calcein loaded target cells of different origins and target lysis was evaluated by measuring Calcein release in the culture media. Cells from G-Rex were used as reference control.

The results show that the modified NK-92® (ha NK, and CD19 t-ha NK, and PD-L1 t-ha NK) cells harvested using 5% Albumin (Human) as wash buffer (post-harvest), induced effective antibody dependent cellular cytotoxicity (ADCC) of tumor cells when coupled with different therapeutic antibodies of clinical grade. Comparable ADCC was observed between reference, pre-harvest and post-harvest samples. See Table 6.

TABLE 6
ADCC Activity of Modified NK-92 ® (haNK, CD19
t-haNK and PD-L1 t-haNK) cells against tumor cells
in combination with therapeutic antibodies of clinical grade

| Modified NK- | | | | |
|---|---|---|---|---|
| 92 ® Effector | | Study Number | Test | % |
| Cells | Tumor Cells | and Date | Sample | Cytotoxicity¹ |
| haNK cells | Ramos cells + | NKSTUDYTP_058 | Reference | 101 ± 5 |
| | Rituxan | 16 May 2018 | Pre-harvest | 97 ± 2 |
| | | | Post-harvest | 93 ± 1 |
| CD19 t-haNK | MDA-MB-231 | NKSTUDYTP_101 | Reference | 50 ± 9 |
| Cells | cells + | 31 Jul. 2018 | Pre-harvest | 42 ± 2 |
| | Avelumab | | Post-harvest | 45 ± 2 |
| PD-L1 t-haNK | MDA-MB-453 | NKSTUDYTP_103 | Reference | 58 ± 1 |
| Cells | cells + | 17 Aug. 2018 | Pre-harvest | 61 ± 2 |
| | Herceptin | | Post-harvest | 70 ± 3 |

¹Modified NK-92 ® cells were centrifuged and harvested in 5% HA. Cells were mixed with the calcein loaded target cells of different origins in the presence of therapeutic antibodies and the target lysis was evaluated by measuring Calcein release in the culture media. Cells from G-Rex were used as reference control.

### Example 4: Viability and Recovery of NK-92® Cells Harvested from Continuous Centrifugation in 5% Albumin (Human)

Modified NK-92® (HER2.ta NK) cells were harvested using continuous centrifugation in either X-VIVO™ 10 or 5% Albumin (Human). Cell viability of pre-harvest and post-harvest samples was examined using a Nucleocounter NC-200 cell counting method and a trypan blue dye-exclusion method. Percent recovery was calculated as the amount of cells recovered from continuous centrifugation/amount of cells entered into continuous centrifugation. The results show that viability of cells harvested from either X-VIVO™ 10 or 5% Albumin (Human) were comparable, 96.7% versus 95.7%. Further, percent cell recovery when harvested using 5% Albumin (Human) was 91.9%, slightly higher than the harvesting using X-VIVO™ 10, which was 89.9%. Further, the viability of the pre-harvest sample and the post-harvest sample was also comparable, indicating that washing cells with 5% Albumin (Human) did not impair cell viability. See Table 7.

TABLE 7
Viability And Recovery Of the Harvested Modified
NK-92 ® (HER2.taNK) Cells

| | | | | % |
|---|---|---|---|---|
| Harvest | Study Date & | Test | % | Re- |
| Media | Number | Sample | Viability | covery |
| X-VIVO ™10 | 22 Jun. 2017 | Pre-harvest | 96.6 | 89.9 |
| | NKSTUDYPRT006 | Post-harvest | 96.7 | |
| 5% ALBUMIN | 27 Jun. 2017 | Pre-harvest | 98 | 91.9 |
| (HUMAN) | NKSTUDYPRT006 | Post-harvest | 95.7 | |

### Example 5: Surface Expression of Modified NK-92® (CD19 t-Ha NK) Cells Harvested from Continuous Centrifugation in 5% Albumin (Human)

Modified NK-92® cells were harvested using continuous centrifugation in 5% Albumin (Human) and the surface expression of the samples were examined using Flow Cytometry based method. Percent surface marker expression was not affected by 5% Albumin (Human) wash as similar amounts of expression was observed on cells from post-harvest, pre-harvest as well as reference cells. See Table 8 and Table 9.

TABLE 8
Phenotyping of the Harvested CD19 t-haNK Cells

| Study Date & | | Cell Surface Marker (%)¹ | | | | | |
|---|---|---|---|---|---|---|---|
| Number | Test Sample | CD3 | CD56 | CD16 | CD54 | NKG2D | NKp30 |
| NKSTUDYTP_100 | Reference | 0.1 | 100 | 99 | 100 | 100 | 99 |
| 20 Jul. 2018 | Pre-harvest | 0.0 | 100 | 96 | 100 | 100 | 98 |
| | Post-harvest | 0.0 | 100 | 95 | 100 | 99 | 95 |

¹Percent (%) of CD19 t-haNK cells positive for the cell surface marker by Flow Cytometry

TABLE 9
CAR-expression on the Harvested Modified
NK-92 ® (CD19 t-haNK) Cells

| Study Date & Number | Test Sample | % CAR+¹ |
|---|---|---|
| NKSTUDYTP_100 | Reference | 99 |
| 20 Jul. 18 | Pre-harvest | 99 |
| | Post-harvest | 99 |

¹Percent of CD19 t-haNK cells positive for CD19.CAR expression determined by Flow Cytometry

It is understood that the examples and embodiments described herein are for illustrative purposes only and that various modifications or changes in light thereof will be suggested to persons skilled in the art and are to be included within the spirit and purview of this application and scope of the appended claims. All publications, sequence accession numbers, patents, and patent applications cited herein are hereby incorporated by reference in their entirety for all purposes.

```
Informal Sequence Listing
High Affinity Variant Immunoglobulin Gamma Fc Region Receptor III-
A nucleic acid sequence (full length form).
SEQ ID NO: 1
ATGTGGCA GCTGCTGCTG CCTACAGCTC TCCTGCTGCT GGTGTCCGCC
GGCATGAGAA CCGAGGATCT GCCTAAGGCC GTGGTGTTCC TGGAACCCCA
GTGGTACAGA GTGCTGGAAA AGGACAGCGT GACCCTGAAG TGCCAGGGCG
CCTACAGCCC CGAGGACAAT AGCACCCAGT GGTTCCACAA CGAGAGCCTG
ATCAGCAGCC AGGCCAGCAG CTACTTCATCGACGCCGCCA CCGTGGACGA
CAGCGGCGAG TATAGATGCC AGACCAACCT GAGCACCCTGAGCGACCCCG
TGCAGCTGGA AGTGCACATC GGATGGCTGC TGCTGCAGGC
CCCCAGATGGGTGTTCAAAG AAGAGGACCC CATCCACCTG AGATGCCACT
CTTGGAAGAA CACCGCCCTGCACAAAGTGA CCTACCTGCA GAACGGCAAG
GGCAGAAAGT ACTTCCACCA CAACAGCGAC TTCTACATCC CCAAGGCCAC
CCTGAAGGAC TCCGGCTCCT ACTTCTGCAG AGGCCTCGTGGGCAGCAAGA
ACGTGTCCAG CGAGACAGTG AACATCACCA TCACCCAGGG
CCTGGCCGTGTCTACCATCA GCAGCTTTTT CCCACCCGGC TACCAGGTGT
CCTTCTGCCT CGTGATGGTGCTGCTGTTCG CCGTGGACAC CGGCCTGTAC
TTCAGCGTGA AAACAAACAT CAGAAGCAGCACCCGGGACT GGAAGGACCA
CAAGTTCAAG TGGCGGAAGG ACCCCCAGGA CAAGTGA
High Affinity Variant Immunoglobulin Gamma Fc Region Receptor III-
A amino acid sequence (full length form). The Val at position 176 is underlined.
SEQ ID NO: 2
Met Trp Gln Leu Leu Leu Pro Thr Ala Leu Leu Leu Leu Val Ser Ala Gly Met Arg Thr Glu
Asp Leu Pro Lys Ala Val Val Phe Leu Glu Pro Gln Trp Tyr Arg Val Leu Glu Lys Asp Ser
Val Thr Leu Lys Cys Gln Gly Ala Tyr Ser Pro Glu Asp Asn Ser Thr Gln Trp Phe His Asn
Glu Ser Leu Ile Ser Ser Gln Ala Ser Ser Tyr Phe Ile Asp Ala Ala Thr Val Asp Asp Ser Gly
Glu Tyr Arg Cys Gln Thr Asn Leu Ser Thr Leu Ser Asp Pro Val Gln Leu Glu Val His Ile Gly
Trp Leu Leu Leu Gln Ala Pro Arg Trp Val Phe Lys Glu Glu Asp Pro Ile His Leu Arg Cys
His Ser Trp Lys Asn Thr Ala Leu His Lys Val Thr Tyr Leu Gln Asn Gly Lys Gly Arg Lys
Tyr Phe His His Asn Ser Asp Phe Tyr Ile Pro Lys Ala Thr Leu Lys Asp Ser Gly Ser Tyr Phe
Cys Arg Gly Leu Val Gly Ser Lys Asn Val Ser Ser Glu Thr Val Asn Ile Thr Ile Thr Gln Gly
Leu Ala Val Ser Thr Ile Ser Ser Phe Phe Pro Pro Gly Tyr Gln Val Ser Phe Cys Leu Val Met
Val Leu Leu Phe Ala Val Asp Thr Gly Leu Tyr Phe Ser Val Lys Thr Asn Ile Arg Ser Ser Thr
Arg Asp Trp Lys Asp His Lys Phe Lys Trp Arg Lys Asp Pro Gln Asp Lys
ER IL-2 nucleic acid sequence
SEQ ID NO: 3
ATGTACCGGATG CAGCTGCTGA GCTGTATCGC CCTGTCTCTG GCCCTCGTGA
CCAACAGCGC CCCTACCAGC AGCAGCACCA AGAAAACCCA GCTGCAGCTG
GAACATCTGC TGCTGGACCTGCAGATGATC CTGAACGGCA TCAACAACTA
CAAGAACCCC AAGCTGACCC GGATGCTGACCTTCAAGTTC TACATGCCCA
AGAAGGCCAC CGAACTGAAA CATCTGCAGT GCCTGGAAGAGGAACTGAAG
CCCCTGGAAG AAGTGCTGAA CCTGGCCCAG AGCAAGAACT
TCCACCTGAGGCCCAGGGAC CTGATCAGCA ACATCAACGT GATCGTGCTG
GAACTGAAAG GCAGCGAGACAACCTTCATG TGCGAGTACG CCGACGAGAC
AGCTACCATC GTGGAATTTC TGAACCGGTGGATCACCTTC TGCCAGAGCA
TCATCAGCAC CCTGACCGGC TCCGAGAAGG ACGAGCTGTGA
ER IL-2 (ER retention signal is underlined) amino acid sequence
SEQ ID NO: 4
Met Tyr Arg Met Gln Leu Leu Ser Cys Ile Ala Leu Ser Leu Ala Leu Val Thr Asn Ser Ala Pro
Thr Ser Ser Ser Thr Lys Lys Thr Gln Leu Gln Leu Glu His Leu Leu Leu Asp Leu Gln Met Ile
Leu Asn Gly Ile Asn Asn Tyr Lys Asn Pro Lys Leu Thr Arg Met Leu Thr Phe Lys Phe Tyr
Met Pro Lys Lys Ala Thr Glu Leu Lys His Leu Gln Cys Leu Glu Glu Glu Leu Lys Pro Leu
Glu Glu Val Leu Asn Leu Ala Gln Ser Lys Asn Phe His Leu Arg Pro Arg Asp Leu Ile Ser
Asn Ile Asn Val Ile Val Leu Glu Leu Lys Gly Ser Glu Thr Thr Phe Met Cys Glu Tyr Ala Asp
Glu Thr Ala Thr Ile Val Glu Phe Leu Asn Arg Trp Ile Thr Phe Cys Gln Ser Ile Ile Ser Thr
Leu Thr Gly Ser Glu Lys Asp Glu Leu
the amino acid sequence of PD-Ll CAR
SEQ ID NO: 5
    1 MDWIWRILFL VGAATGAHSA QPANIQMTQS PSSVSASVGD RVTITCRASQ DISRWLAWYQ
  61 QKPGKAPKLL IYAASSLQSG VPSRFSGSGS GTDFALTISS LQPEDFATYY CQQADSRFSI
121 TFGQGTRLEI KGGGGSGGGG SGGGGSGGGG SEVQLVQSGG GLVQPGGSLR LSCAASGFTF
181 SSYSMNWVRQ APGKGLEWVS YISSSSSTIQ YADSVKGRFT ISRDNAKNSL YLQMNSLRDE
241 DTAVYYCARG DYYYGMDVWG QGTTVTVSSA AALSNSIMYF SHFVPVFLPA KPTTTPAPRP
301 PTPAPTIASQ PLSLRPEACR PAAGGAVHTR GLDFACFWVL VVVGGVLACY SLLVTVAFII
361 FWVRLKIQVR KAAITSYEKS DGVYTGLSTR NQETYETLKH EKPPQ
the amino acid sequence of CD19 CAR
SEQ ID NO: 6
    1 MDWIWRILFL VGAATGAHSA QPADIQMTQT TSSLSASLGD RVTISCRASQ DISKYLNWYQ
  61 QKPDGTVKLL IYHTSRLHSG VPSRFSGSGS GTDYSLTISN LEQEDIATYF CQQGNTLPYT
121 FGGGTKLELK RGGGGSGGGG SGGGGSGGGG SEVQLQQSGP GLVAPSQSLS VTCTVSGVSL
181 PDYGVSWIRQ PPRKGLEWLG VIWGSETTYY NSALKSRLTI IKDNSKSQVF LKMNSLQTDD
241 TAIYYCAKHY YYGGSYAMDY WGQGTTVTVS SAAALSNSIM YFSHFVPVFL PAKPTTTPAP
301 RPPTPAPTIA SQPLSLRPEA CRPAAGGAVH TRGLDFACFW VLVVVGGVLA CYSLLVTVAF
361 IIFWVRLKIQ VRKAAITSYE KSDGVYTGLS TRNQETYETL KHEKPPQ
the amino acid sequence of Her2 CAR
SEQ ID NO: 7
MDWIWRILFLVGAATGAHSAQPADIQMTQSPSSLSASVGDRVTITCRASQDVNTAVAWYQQK
PGKAPKLLIYSASFLYSGVPSRFSGSRSGTDFTLTISSLQPEDFATYYCQQHYTTPPTFGQG
TKVEIKSSGGGGSGGGGSGGGGSGGGGSGGSEVQLVESGGGLVQPGGSLRLSCAASGFNIKD
TYIHWVRQAPGKGLEWVARTYPTNGYTRYADSVKGRFTISADTSKNTAYLQMNSLRAEDTAV
YYCSRWGGDGFYAMDYWGQGTLVTVSS
```

What is claimed is:

1. A method of harvesting NK-92® cells comprising collecting NK-92® cells from a cell culture and washing the collected NK-92® cells by resuspending the cells in a buffer comprising 1-5% albumin.

2. The method of claim 1, wherein the collecting NK-92® cells comprising centrifuging the NK-92® cells in the cell culture.

3. The method of claim 1, further comprising placing the washed NK-92® cells in an infusion bag.

4. The method of claim 1, wherein the wash is performed by centrifuging the cells and then resuspending the cells in the wash buffer.

5. The method of claim 1, wherein the wash is performed at least three times.

6. The method of claim 1, wherein the wash is performed 4-6 times.

7. The method of claim 6, wherein the method recovers at least 80% of the NK-92® cells.

8. The method of claim 1, wherein the viability of the harvested cells is at least 90%.

9. The method of claim 1, wherein the NK-92® cells that have been harvested have substantially the same cytotoxicity and/or viability as control NK-92® cells that have not been harvested.

10. The method of claim 1, wherein the NK-92® cells that have been harvested have substantially the same cytotoxicity and/or viability as the NK-92® cells before harvesting.

11. The method of claim 1, wherein the NK-92® cells that have been harvested have a cytotoxicity of 80-100% on K562 cells.

12. The method of claim 1, wherein the buffer contains 2-5% albumin.

13. The method of claim 1, wherein the buffer contains 3-5% albumin.

14. The method of claim 1, wherein the buffer contains 5% albumin.

15. The method of claim 1, wherein the buffer lacks sugar.

16. The method of claim 1, wherein the buffer lacks dextran.

17. The method of claim 4, wherein the centrifugation is by continuous centrifugation.

18. The method of claim 1, wherein the albumin is human albumin, or human serum derived albumin, or human plasma derived albumin.

19. The method of claim 1, wherein the NK-92® cells express a cytokine, Fc Receptor, a chimeric antigen receptor, or a combination thereof.

20. The method of claim 19, wherein the chimeric antigen receptor is a receptor for HER2, CD19 or PD-L1.

21. The method of claim 19, wherein the chimeric antigen receptor is a receptor for any of the tumor specific antigens listed in Table 1.
